(19) United States
(12) Patent Application Publication
(10) Pub. No.: US 2014/0100474 A1
(43) Pub. Date: Apr. 10, 2014

(54) Palpatory Diagnostic System and Method

(71) Applicant: Ira D Shandles, Tampa, FL (US)

(72) Inventor: Ira D Shandles, Tampa, FL (US)

(21) Appl. No.: 14/101,553

(22) Filed: Dec. 10, 2013

Related U.S. Application Data
(63) Continuation-in-part of application No. 13/134,101, filed on May 27, 2011.

(51) Int. Cl. A61B 5/00

(52) U.S. Cl. CPC A61B 5/0053; A61B 5/4029
USPC 600/557

(57) ABSTRACT

A generally cylindrical housing has proximal and distal ends and is formed with a length and a diameter. A cylindrical recess is formed in the proximal end. The cylindrical recess has an interior shoulder. A cone is formed at the distal end. An axial passageway extends though the cone. A hollow interior is in the housing forming a chamber in communication with the axial passageway. A cylindrical identifying component is positioned within the recess in contact with the shoulder. A marking component is positioned within the axial passageway and has an extent projecting distally of the distal end. A marking fluid is located within the chamber.

### RELATED APPLICATION

The present application is a continuation-in-part of pending U.S. patent application Ser. No. 13/134,101 filed May 27, 2011, the subject matter of which application is incorporated herein by reference.

### BACKGROUND OF THE INVENTION

1. Field of the Invention

The present invention relates to a palpatory diagnostic system and method and more particularly pertains to touching an identifying component to a patient for identifying neuromas and nerve entrapments and then marking identified neuromas and nerve entrapments.

2. Description of the Prior Art

The use of diagnostic devices is known in the prior art. More specifically, diagnostic devices previously devised and utilized for the purpose of facilitating patient treatment are known to consist basically of familiar, expected, and obvious structural configurations, notwithstanding the myriad of designs encompassed by the crowded prior art which has been developed for the fulfillment of countless objectives and requirements.

By way of example, U.S. Pat. No. 6,616,619 to Fusco relates to a diagnostic and therapeutic method for identification and treatment of postural and disturbances. United States Patent Publication Number 2008/0193905 to Fong relates to automated testing for palpating diabetic foot patient. Lastly, U.S. Pat. No. 7,727,170 to Warner discloses an elastic hysteretic palpatory training apparatus and method of use thereof.

While these devices fulfill their respective, particular objectives and requirements, the aforementioned patents do not describe a palpatory diagnostic system and method that allows touching an identifying component to a patient for identifying neuromas and nerve entrapments and then marking the identified neuromas and nerve entrapments confining the location to a treatable area in direct proximity to a tumor increasing the success rate of treatment.

In this respect, the palpatory diagnostic system and method according to the present invention substantially departs from the conventional concepts and designs of the prior art, and in doing so provides an apparatus primarily developed for the purpose of touching an identifying component to a patient for identifying neuromas and nerve entrapments and then marking the identified neuromas and nerve entrapments confining the location to a treatable area in direct proximity to a tumor increasing the success rate of treatment.

Therefore, it can be appreciated that there exists a continuing need for a new and improved palpatory diagnostic system and method which can be used for touching an identifying component to a patient for precisely identifying neuromas and nerve entrapments and then marking the identified neuromas and nerve entrapments for treatment that is diagnostic, durable economical and consistent. In this regard, the present invention substantially fulfills this need.

### SUMMARY OF THE INVENTION

In view of the foregoing disadvantages inherent in the known types of diagnostic devices now present in the prior art, the present invention provides an improved palpatory diagnostic system and method. As such, the general purpose of the present invention, which will be described subsequently in greater detail, is to provide a new and improved palpatory diagnostic system and method and method which has all the advantages of the prior art and none of the disadvantages.

To attain this, the present invention essentially comprises a palpatory diagnostic system for identifying neuromas and nerve entrapments of body parts, for marking identified neuromas and nerve entrapments, confining the location to a circular treatable area, the centre of which is in direct proximity to a tumor, the identifying and marking being done in a safe, comfortable, convenient, efficient and economical manner. The marking and identifying diagnostically increasing the success rate of treatment.

The system comprises, in combination, a generally cylindrical housing having proximal and distal ends and formed with a length and a diameter with an axial passageway. The proximal end has a cylindrical identifying component positioned within the proximal end and protruding from the proximal end. The identifying component is fabricated of a resilient elastomer chosen from the class of medical grade resilient elastomers including silicone, and vinyl and having a having a Shore hardness of between 50 to 60. The proximal end thereby constitutes an identifying end.

The distal end includes a marking component positioned within the axial passageway and having an extent projecting distally of a cone formed at the distal end. In one embodiment, An axial passageway extends though the cone and the interior of the housing is hollow forming a cylindrical chamber in communication with the axial passageway. A porous tip is positioned within the axial passageway at the distal end of the housing. The porous tip having a minority extent projecting distally of the distal end of the housing. The porous tip has a majority extent within the axial passageway in fluid communication with the chamber. The porous tip is fabricated from a semi-rigid porous material preferably chosen from the class of semi-rigid porous materials including felt, nylon and ceramic. The distal end of the housing thereby constituting a marking end.

A marking fluid is located within the chamber and the axial passageway and in contact with the porous tip when the system is in a marking orientation with the proximal section above the distal section. The marking fluid is adapted to be dispensed from the chamber and axial passageway through the porous tip when contacting identified neuromas and nerve entrapments to thereby mark an identified part of a patient's body. The marking fluid is chosen from dermatologically acceptable indelible marking fluids including gentian violet ink.

One feature of the invention is a cover for the distal end to prevent unintended markings and protect the marking end. The cover is formed in a generally cylindrical configuration. The cover has an open end and a closed end. The cover is removably received on the distal end of the housing. The cover is adapted to be frictionally retained on the distal end of the housing during storage and transportation. The cover is adapted to be removed from the distal end of the housing during operation and use for marking purposes. The cover is fabricated of a material the same as the housing.

Another feature of the invention is a clip adjacent to the proximal end for removably coupling the housing to a physician user.

The invention also includes a system constructed in accordance with an alternate embodiment of the invention which includes a replaceable ink cartridge utilizing a nib with a rotatable ball as the marking component and releasably coupled proximal and distal end components. A further alternate embodiment includes the identifying and marking components of the primary embodiment and a supporting rod positioned within the identifying end to add stability and restrict movement of the identifying component. The invention also includes the method of using the system.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims attached.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved palpatory diagnostic system and method which has all of the advantages of the prior art diagnostic devices and none of the disadvantages.

It is another object of the present invention to provide a new and improved palpatory diagnostic system and method which may be easily and efficiently manufactured and marketed.

It is further object of the present invention to provide a new and improved palpatory diagnostic system and method which is of durable and reliable constructions.

An even further object of the present invention is to provide a new and improved palpatory diagnostic system and method which is susceptible of a low cost of manufacture with regard to both materials and labor, and which accordingly is then susceptible of low prices of sale to the consuming public, thereby making such palpatory diagnostic system and method economically available to the buying public.

Even still another object of the present invention is to provide a palpatory diagnostic system and method for touching an identifying component to a patient for identifying neuromas and nerve entrapments and then marking the identified neuromas and nerve entrapments that is diagnostic, durable and consistent.

Lastly, it is an object of the present invention to provide a new and improved palpatory diagnostic system and method for touching an identifying component to a patient for identifying neuromas and nerve entrapments and then marking the identified neuromas and nerve entrapments confining the location to a treatable area in direct proximity to a tumor increasing the success rate of treatment.

These together with other objects of the invention, along with the various features of novelty which characterize the invention, are pointed out with particularity in the claims annexed to and forming a part of this disclosure.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a front elevational view of a palpatory diagnostic system constructed in accordance with the principles of the present invention.

FIG. 2 is an exploded view of a palpatory diagnostic system constructed in accordance with the principles of the present invention.

FIG. 3 is an exploded view of a palpatory diagnostic system constructed in accordance with the principles of an alternate embodiment of the present invention.

FIG. 4 is a cross sectional view of an alternate embodiment of a palpatory diagnostic system taken along line 4-4 of FIG. 3.

FIG. 5 is a front elevational view of a palpatory diagnostic system constructed in accordance with an alternate embodiment of the invention.

FIG. 6 is an exploded view of a palpatory diagnostic system constructed in accordance with an alternate embodiment of the invention.

### The features of the invention shown throughout the various figures and embodiments are representative of the invention.

### DESCRIPTION OF THE PREFERRED EMBODIMENT

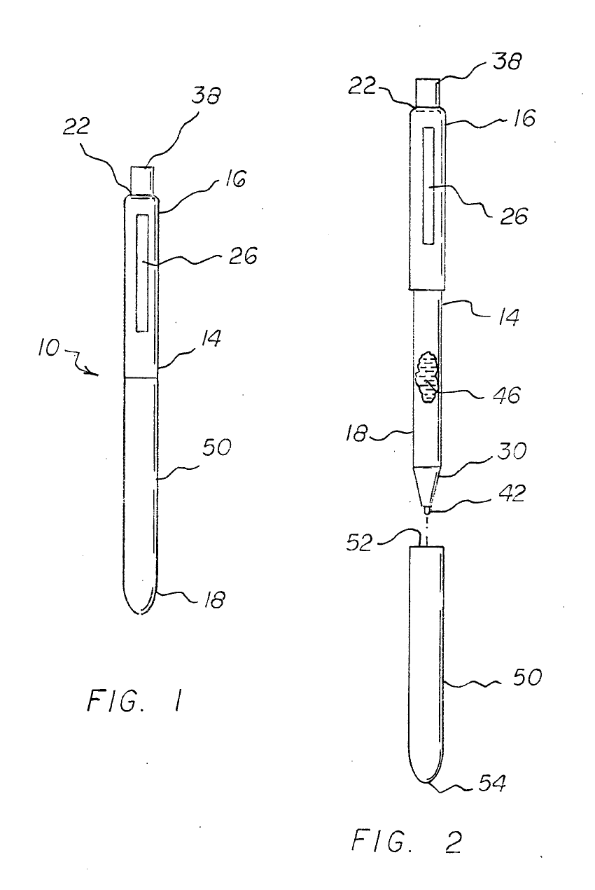

With reference now to the drawings, and in particular to FIGS. 1-2 thereof, the preferred embodiment of the new and improved palpatory diagnostic system and method embodying the principles and concepts of the present invention and generally designated by the reference numeral 10 will be described.

The present invention, the palpatory diagnostic system and method 10 is comprised of a plurality of components. Such components in their broadest context include a housing, an identifying component, a marking component and a marking fluid. Such components are individually configured and correlated with respect to each other so as to attain the desired objective.

The primary embodiment invention is thus a palpatory diagnostic system 10 for identifying neuromas and nerve entrapments of body parts and for marking identified neuromas and nerve entrapments, confining the location to a circular treatable area, the centre of which is in direct proximity to a tumor thereby diagnostically pinpointing the exact location of treatment. The identifying, marking and confinement of the location is done in a safe, consistent, comfortable, convenient, efficient and economical manner. The system comprises, in combination, a housing 14 having a proximal end 16 and a distal end 18, the housing having a length and an exterior diameter.

Next provided is an enlarged cylindrical recess 22 in the proximal end. The enlarged cylindrical recess has a diameter between 50 and 75 percent of the exterior diameter of the housing. The enlarged cylindrical recess has an interior shoulder. The enlarged cylindrical recess has a length extending between the interior shoulder and the proximal end of the proximal section. The proximal end thereby constitutes an identifying end. A clip 26 adjacent to the proximal end functions for removably coupling the housing to a physician user.

A cone 30 is formed at the distal end. An axial passageway extends though the cone. A hollow interior is in the housing. The hollow interior forms a cylindrical chamber in communication with the axial passageway. The distal end of the housing thereby constitutes a marking end. The housing is fabricated of a rigid material chosen from the class of rigid materials including metal, plastic and composite. An identifying component 38 is in a cylindrical configuration and has a length and a diameter. The diameter of the identifying component is essentially equal to the diameter of the enlarged recess at the proximal end of the housing. The length of the identifying component is between 180 and 220 percent of the length of the enlarged opening. The identifying component is positioned within the enlarged recess in contact with the shoulder, the shoulder forming an abutment surface for the identifying component, and protruding a minimum length of 0.38 inches. The identifying component has a diameter between 0.4 and 0.45 inches. The identifying component is fabricated of a resilient elastomer chosen from the class of medical grade resilient elastomers including silicone, and vinyl and having a having a Shore hardness of 50 to 60.

A porous tip 42 is positioned within the axial passageway at the distal end of the housing. The porous tip has a minority extent projecting distally of the distal end of the housing. The porous tip has a majority extent within the axial passageway in fluid communication with the chamber. The porous tip is fabricated from a semi-rigid porous material preferably chosen from the class of semi-rigid porous materials including felt, nylon and ceramic.

A marking fluid 46 is located within the chamber and the axial passageway and in contact with the porous tip when the system is in a marking orientation with the proximal section above the distal section. The marking fluid is adapted to be dispensed from the chamber and axial passageway through the porous tip when contacting identified neuromas and nerve entrapments to thereby mark an identified part of a patient's body. The marking fluid is chosen from dermatologically acceptable indelible marking fluids including gentian violet ink.

Lastly, a cover 50 is in a generally cylindrical configuration. The cover has an open end 52 and a closed end 54. The cover is removably received on the distal end of the housing. The cover is adapted to be frictionally retained on the distal end of the housing during storage and transportation. The cover is adapted to be removed from the distal end of the housing during operation when used for marking purposes. The cover is fabricated of a material the same as the housing.

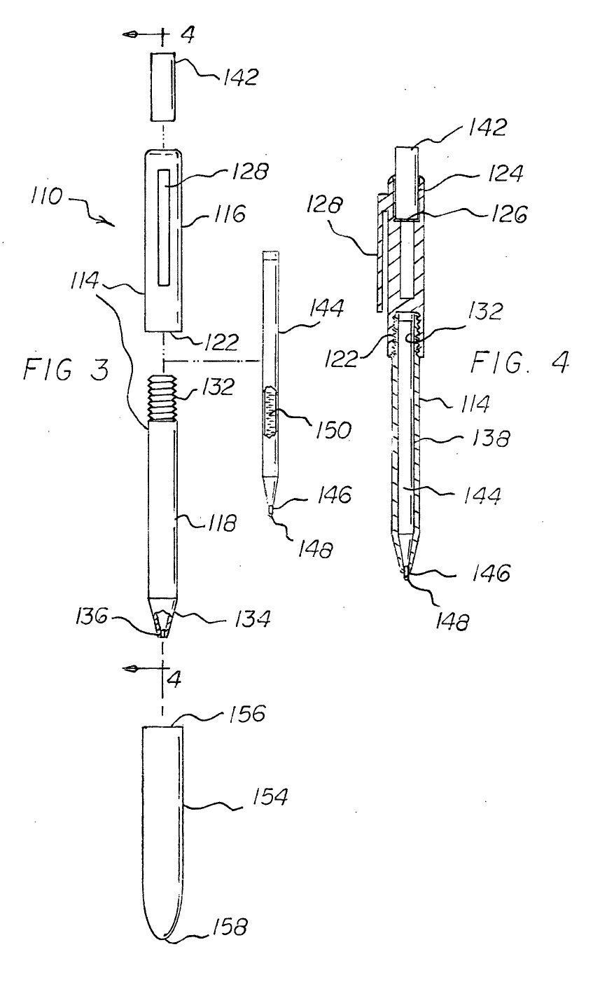

The invention also includes an alternate embodiment of the invention. As shown in FIGS. 3-4, the alternate embodiment is a palpatory diagnostic system 110 for identifying neuromas and nerve entrapments of body parts and for marking identified neuromas and nerve entrapments, confining the location to a circular treatable area, the centre of which is in direct proximity to a tumor thereby diagnostically pinpointing the exact location of treatment. The identifying and the marking being done in a safe, consistent, comfortable, convenient, efficient and economical manner. The system comprises, in combination, a housing 114 having a proximal section 116 and a distal section 118. Each of the sections is formed in a generally cylindrical configuration. Each of the sections has a length and an exterior diameter.

The proximal section has a distal end formed with female threads 122. The proximal section has a proximal end formed with an enlarged cylindrical recess 124. The enlarged cylindrical recess has a diameter between 50 and 75 percent of the exterior diameter of the proximal section. The enlarged cylindrical recess has an interior shoulder 126. The enlarged cylindrical recess has a length extending between the interior shoulder and the proximal end of the proximal section. The proximal end of the proximal section thereby constitutes an identifying end. A clip 128 is formed on the proximal section for removably coupling the housing to a physician user.

The distal section has a proximal end formed with male threads 132 adapted to releasably couple with the female threads of the proximal section. The distal section has a distal end formed as a cone 134 with an axial passageway 136. The distal section is formed with a hollow interior forming cylindrical chamber 138 in communication with the axial passageway and extending to the proximal end of the distal section. The distal end of the distal section thereby constituting a marking end. The housing is fabricated of a rigid material chosen from the class of rigid materials including metal, plastic and composite.

An identifying component 142 is in a cylindrical configuration and has a length and a diameter. The diameter of the identifying component is essentially equal to the diameter of the enlarged recess at the proximal end of the proximal section. The length of the identifying component is between 180 and 220 percent of the length of the enlarged opening. The identifying component is positioned within the enlarged recess in contact with the shoulder, the shoulder forming an abutment surface for the identifying component, and protruding a minimum length of 0.38 inches. The identifying component having a diameter of 0.45 inches. The identifying component is fabricated of a resilient elastomer chosen from the class of medical grade resilient elastomers including silicone, and vinyl and having a having a Shore hardness of 50 to 60.

The distal end has a marking component, the marking component includes a replaceable cartridge 144 located within the axial chamber of the housing having a minority extent extending distally of the distal end cone having a generally cylindrical housing with a distal and proximal end. A nib 146 is at the distal end. An axial passageway extends through the nib. A hollow interior is in the housing forming a chamber in communication with the axial passageway. A rotatable ball 146 is positioned within the axial passageway at the distal end of the nib, the rotatable ball having a minority extent projecting distally of the distal end of the nib. The rotatable ball has a majority extent within the axial passageway in fluid communication with the chamber. The rotatable ball is fabricated from a hard liquid impervious preferably chosen from the class of hard liquid impervious including brass, steel and tungsten carbide.

A marking fluid 150 is located within the chamber and the axial passageway and in contact with the rotatable ball when the system is in a marking orientation with the proximal section above the distal section. The marking fluid is adapted to be dispensed from the chamber and axial passageway as the ball rotates when contacting identified neuromas and nerve entrapments to thereby mark an identified part of a patient's body. The marking fluid is chosen from dermatologically acceptable indelible marking fluids including gentian violet ink.

Lastly, a cover 154 is in a generally cylindrical configuration. The cover has an open end 156 and a closed end 158. The cover is removably received on the distal section of the housing. The cover is adapted to be frictionally retained on the distal section of the housing during storage and transportation. The cover is adapted to be removed from the distal section of the housing during operation and use for marking purposes.

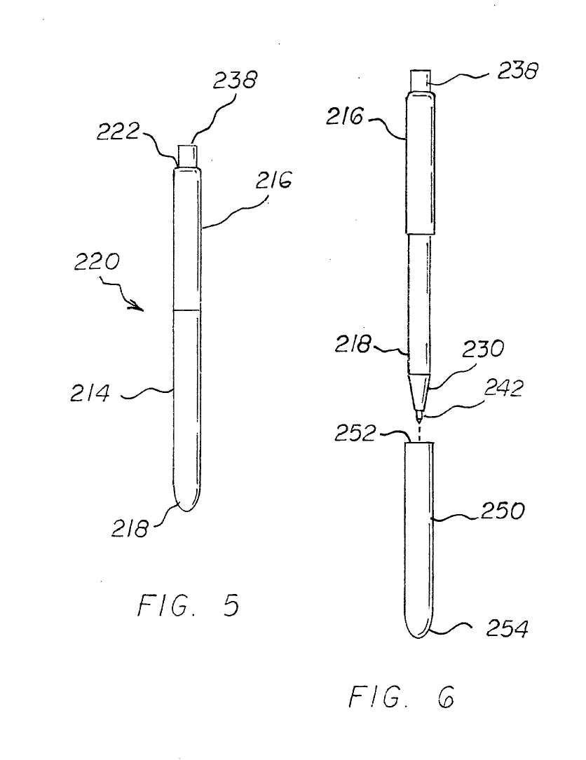

A third embodiment is illustrated in FIGS. 5-6 and is a palpatory diagnostic system 220 for identifying neuromas and nerve entrapments of body parts and for marking identified neuromas and nerve entrapments, confining the location to a circular treatable area, the centre of which is in direct proximity to a tumor thereby diagnostically pinpointing the exact location of treatment. The identifying, marking and confinement of the location is done in a safe, consistent, comfortable, convenient, efficient and economical manner. The system comprises, in combination, a housing 214 having a proximal end 216 and a distal end 218, the housing having a length and an exterior diameter.

Next provided is an enlarged cylindrical recess 222 in the proximal end. The enlarged cylindrical recess has a diameter between 50 and 75 percent of the exterior diameter of the housing. The enlarged cylindrical recess has an interior shoulder. The enlarged cylindrical recess has a length extending between the interior shoulder and the proximal end of the proximal section. The proximal end thereby constitutes an identifying end.

A cone 230 is formed at the distal end. An axial passageway extends though the cone. A hollow interior is in the housing. The hollow interior forms a cylindrical chamber in communication with the axial passageway. The distal end of the housing thereby constitutes a marking end. The housing is fabricated of a rigid material chosen from the class of rigid materials including metal, plastic and composite. An identifying component 238 is in a cylindrical configuration and has a length and a diameter. The diameter of the identifying component is essentially equal to the diameter of the enlarged recess at the proximal end of the housing. The length of the identifying component is between 180 and 220 percent of the length of the enlarged opening. The identifying component is positioned within the enlarged recess in contact with the shoulder, the shoulder forming an abutment surface for the identifying component, and protruding a minimum length of 0.38 inches. The identifying component is fabricated of a resilient elastomer chosen from the class of medical grade resilient elastomers including silicone, and vinyl and having a having a Shore hardness of 50 to 60.

A porous tip 242 is positioned within the axial passageway at the distal end of the housing. The porous tip has a minority extent projecting distally of the distal end of the housing. The porous tip has a majority extent within the axial passageway in fluid communication with the chamber. The porous tip is fabricated from a semi-rigid porous material preferably chosen from the class of semi-rigid porous materials including felt, nylon and ceramic.

A marking fluid is located within the chamber and the axial passageway and in contact with the porous tip when the system is in a marking orientation with the proximal section above the distal section. The marking fluid is adapted to be dispensed from the chamber and axial passageway through the porous tip when contacting identified neuromas and nerve entrapments to thereby mark an identified part of a patient's body. The marking fluid is chosen from dermatologically acceptable indelible marking fluids including gentian violet ink.

Lastly, a cover 250 is formed in a generally cylindrical configuration. The cover has an open end 252 and a closed end 254. The cover is removably received on the distal end of the housing. The cover is adapted to be frictionally retained on the distal end of the housing during storage and transportation. The cover is adapted to be removed from the distal end of the housing during operation when used for marking purposes. The cover is fabricated of a material the same as the housing.

The invention also includes the method of using the systems as described above. The method is a palpatory diagnostic method for identifying neuromas and nerve entrapments of the body parts, confining the location to a circular treatable area, the centre of which is in direct proximity to a tumor. The identifying and the marking being is done in a safe, consistent, comfortable, convenient, efficient and economical manner, the method comprising, in combination, the steps as follows:

a. providing a housing having a proximal end and a distal end, the housing having a length and an exterior diameter, the housing being fabricated of a rigid material chosen from the class of rigid materials including metal, plastic and composite;

b. providing an identifying component positioned within the proximal end and protruding a minimum length of 0.38 inches. The identifying component having a diameter of 0.4 to 0.45 inches. The identifying component is fabricated of a resilient elastomer chosen from the class of medical grade resilient elastomers including silicone, and vinyl and having a having a Shore hardness of 50 to 60;

c. providing a clip adjacent to the proximal end for removably coupling the housing to a physician user;

d. forming a cone at the distal end with an axial passageway extending though the cone, a hollow interior in the housing, the hollow interior forming a cylindrical chamber in communication with the axial passageway, the distal end of the housing thereby constituting a marking end;

e. locating a marking component within the axial chamber of the housing having a minority extent extending distally of the distal end cone;

f. providing a marking fluid being located in contact with the marking component, the marking fluid being chosen from dermatologically acceptable indelible marking fluids including gentian violet ink;

g. with the proximal end of the housing beneath the distal end of the housing, touching the identifying component to body part of a patient by a physician observing the body part of the patient in an effort to identify neuromas and nerve entrapments of body parts;

h. while continuing to observe the body part of the patient, rotating the housing 180 degrees; and

i. with the proximal end of the housing above the distal end of the housing, touching the marking component to a body part of a patient by a physician observing the body part of the patient in an effort to mark the location of identified neuromas and nerve entrapments of the body parts, confining the location to a circular treatable area, the centre of which is in direct proximity to a tumor.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A palpatory diagnostic system comprising:
a generally cylindrical housing having proximal and distal ends and being formed with a length and a diameter;
a cylindrical recess in the proximal end, the cylindrical recess having an interior shoulder;
a cone formed at the distal end, an axial passageway extending though the cone, a hollow interior in the housing forming a chamber in communication with the axial passageway;
a cylindrical identifying component positioned within the recess in contact with the shoulder and protruding a minimum length of 0.38 inches, the identifying component is fabricated of a resilient elastomer chosen from the class of medical grade resilient elastomers including silicone, and vinyl and having a having a Shore hardness of 50 to 60;
a marking component positioned within the axial passageway and having an extent projecting distally of the distal end; and
a marking fluid located within the chamber.

2. The system as set forth in claim 1 wherein the identifying component has a diameter between 0.4 and 0.45 inches.

3. The system as set forth in claim 1 and further including:
a cover in a generally cylindrical configuration, the cover having an open end and a closed end, the cover being removably received on the distal end of the housing, the cover adapted to be frictionally retained on the distal end of the housing during storage and transportation, the cover adapted to be removed from the distal end of the housing during operation and use for marking purposes.

4. The system as set forth in claim 1 wherein the housing is formed of a distal section and a proximal section with screw threads coupling the distal and proximal sections.

5. The system as set forth in claim 1 wherein the housing is fabricated of a rigid material chosen from the class of rigid materials including metal, plastic and composite.

6. The system as set forth in claim 1 wherein the marking component is a ball fabricated of a rigid, liquid impervious material chosen from the class of rigid impervious materials including brass, steel and tungsten carbide.

7. The system set forth in claim 1 wherein the marking component is a conical tip fabricated of compressed porous material chosen from a class of porous materials including felt, nylon and ceramic.

8. The system as set forth in claim 1 wherein the marking fluid is chosen from the class of dermatologically acceptable indelible marking fluids including gentian violet ink.

9. A palpatory diagnostic system for identifying neuromas and nerve entrapments of body parts and for marking identified neuromas and nerve entrapments, the identifying and the marking being done in a safe, comfortable, convenient, efficient and economical manner, the system comprising, in combination:
a housing having a proximal section and a distal section , each of the sections being formed in a generally cylindrical configuration, each of the sections having a length and an exterior diameter;
the proximal section having a distal end formed with female threads, the proximal section having a proximal end formed with an enlarged cylindrical recess, the enlarged cylindrical recess having a diameter between 50 and 75 percent of the exterior diameter of the proximal section, the enlarged cylindrical recess having an interior shoulder, the enlarged cylindrical recess having a length extending between the interior shoulder and the proximal end of the proximal section, the proximal end of the proximal section thereby constituting an identifying end, a clip formed on the proximal section for removably coupling the housing to a physician user;
the distal section having a proximal end formed with male threads adapted to releasably couple with the female threads of the proximal section, the distal section having a distal end formed as a cone with an axial passageway, the distal section being formed with a hollow interior forming cylindrical chamber in communication with the axial passageway and extending to the proximal end of the distal section, the distal end of the distal section thereby constituting a marking end, the housing being fabricated of a rigid material chosen from the class of rigid materials including metal, plastic and composite;
an identifying component in a cylindrical configuration and having a length and a diameter, the diameter of the identifying component being essentially equal to the diameter of the enlarged recess at the proximal end of the proximal section, the length of the identifying component being between 180 and 220 percent of the length of the enlarged opening, the identifying component being positioned within the enlarged recess in contact with the shoulder with the shoulder forming an abutment surface for the identifying component, the identifying component having a diameter between 0.40 and 0.45 inches, the identifying component having an axial length between 0.40 and 0.60 inches and protruding a minimum length of 0.38 inches, the identifying component being fabricated of a medical grade resilient elastomer chosen from the class of medical grade resilient elastomers including silicone, and vinyl and having a having a Shore hardness of 50 to 60;
a marking component, the marking component including a replaceable cartridge located within the axial chamber of the housing having a minority extent extending distally of the distal end cone having a generally cylindrical housing with a distal and proximal end, a nib at the distal end, an axial passageway extending through the nib, a hollow interior in the housing forming a chamber in communication with the axial passageway, a rotatable ball positioned within the axial passageway at the distal end of the nib, the rotatable ball having a minority extent projecting distally of the distal end of nib, the rotatable ball having a majority extent within the axial passageway in fluid communication with the chamber, the rotatable ball being fabricated from a hard liquid impervious preferably chosen from the class of hard liquid impervious including brass, steel and tungsten carbide; a marking fluid, the marking fluid being located within the chamber and the axial passageway and in contact with the rotatable ball when the system is in a marking orientation with the proximal section above the distal section, the marking fluid adapted to be dispensed from the chamber and axial passageway as the ball rotates when contacting identified neuromas and nerve entrapments to thereby mark an identified part of a patient's body, the marking fluid being chosen from dermatologically acceptable indelible marking fluids including gentian violet ink; and
a cover in a generally cylindrical configuration, the cover having an open end and a closed end, the cover being removably received on the distal section of the housing, the cover adapted to be frictionally retained on the distal section of the housing during storage and transportation, the cover adapted to be removed from the distal section of the housing during operation and use for marking purposes.

10. A palpatory diagnostic method for identifying neuromas and nerve entrapments of body parts, confining the location to a circular treatable area, the centre of which is in direct proximity to a tumor and for marking the treatment zone of identified neuromas and nerve entrapments, the identifying and the marking being done in a safe, consistent, comfortable, convenient, efficient and economical manner, the method comprising, in combination:
providing a housing having a proximal end and a distal end, the housing having a length and an exterior diameter, the housing being fabricated of a rigid material chosen from the class of rigid materials including metal, plastic and composite;
providing an identifying component positioned within the proximal end and protruding a minimum length of 0.38 inches. The identifying component having a diameter of 0.4 to 0.45 inches. The identifying component is fabricated of a resilient elastomer chosen from the class of medical grade resilient elastomers including silicone, and vinyl and having a having a Shore hardness of 50 to 60;
providing a clip adjacent to the proximal end for removably coupling the housing to a physician user;
forming a cone at the distal end with an axial passageway extending though the cone, a hollow interior in the housing, the hollow interior forming a cylindrical chamber in communication with the axial passageway, the distal end of the housing thereby constituting a marking end;
locating a marking component within the axial chamber of the housing having a minority extent extending distally of the distal end cone;
providing a marking fluid being located in contact with the marking component, the marking fluid being chosen from dermatologically acceptable indelible marking fluids including gentian violet ink;
with the proximal end of the housing beneath the distal end of the housing, touching the identifying component to a body part of a patient by a physician observing the body part of the patient in an effort to identify neuromas and nerve entrapments of the body part;
while continuing to observe the body part of the patient, rotating the housing 180 degrees;
with the proximal end of the housing above the distal end of the housing, touching the marking component to a body part of a patient by a physician observing the body part of the patient in an effort to mark the location of identified neuromas and nerve entrapments of the body parts, confining the location to a circular treatable area, the centre of which is in direct proximity to a tumor.
